(19) United States
(12) Patent Application Publication
(10) Pub. No.: US 2023/0149360 A1
(43) Pub. Date: May 18, 2023

(54) ALPHA-2 ADRENERGIC RECEPTOR AGONISTS FOR THE PREVENTION AND/OR THE TREATMENT OF SPLEEN DISORDERS

(71) Applicant: UNIVERSITÉ CATHOLIQUE DE LOUVAIN, Louvain la Neuve (BE)

(72) Inventors: Benoît VAN DEN EYNDE, Genval (BE); Jingjing ZHU, Kapellen (BE)

(73) Assignee: UNIVERSITÉ CATHOLIQUE DE LOUVAIN, Louvain la Neuve (BE)

(21) Appl. No.: 17/920,560

(22) PCT Filed: Apr. 21, 2021
(86) PCT No.: PCT/EP2021/060441

§ 371 Date: Oct. 21, 2022

(30) Foreign Application Priority Data
Apr. 21, 2020 (WO) 20170703.1

(51) Int. Cl. A61K 31/4168; A61K 45/06; A61P 35/00; A61K 31/155; A61P 37/00

(52) U.S. Cl. CPC A61K 31/4168; A61K 45/06; A61P 35/00; A61K 31/155; A61P 37/00

(57) ABSTRACT

The present invention relates to the therapeutic use of alpha-2 adrenergic receptor agonists in the prevention and/or treatment of spleen disorders, in particular splenomegaly. The inventors showed that clonidine, guanabenz and romifidine efficiently reduced the spleen weight of animal models with cancer-induced splenomegaly, immunization-induced splenomegaly or splenomegaly induced by myelofibrosis.

### FIELD OF INVENTION

The present invention relates to the therapeutic use of alpha-2 adrenergic receptor agonists in the prevention and/or the treatment of spleen disorders, in particular splenomegaly. In particular, clonidine, guanabenz and romifidine, three alpha-2 adrenergic receptor agonists, are capable of efficiently reducing the spleen weight of individuals with cancer-induced splenomegaly, immunization-induced splenomegaly, or splenomegaly induced by myelofibrosis.

### BACKGROUND OF INVENTION

The spleen is a fist-sized organ, which is located in the upper left side of the abdomen, next to the stomach and behind the left ribs. The spleen plays an important role of the immune system, e.g., by fighting invading germs in the blood or by controlling the level of blood cells, i.e., the white blood cells, red blood cells and platelets. In addition, the spleen filters the blood and removes any old or damaged red blood cells. Finally, it has been suggested that the spleen may represent a constitutive part of the sympathetic nervous system during stressful situations Bakovic et al. (Journal of Physiology and Pharmacology, 2013, Vol. 64(5), p. 649-655).

In practice, damaged spleen may be removed, as a surgical procedure named splenectomy. In that case, the liver may compensate many functions of the removed spleen.

Splenomegaly, also known as “enlarged spleen”, is a medical term intended to refer to a pathological enlargement of the spleen, which can occur as a result of many infections, tumors, and hereditary or acquired conditions of the blood leading to hemolysis. The normal spleen weighs approximately 200 g and is usually impalpable; in disease states it may enlarge to become about 2 kg at most. The enlarged spleen may also become overactive, devouring too many blood cells and producing other symptoms.

The spleen may be enlarged in a range of conditions, such as, e.g., cancer; liver disorders such as cirrhosis, portal hypertension and portal or splenic vein thrombosis or compression, which leads to increase in the blood pressure inside the splenic vasculature; cystic fibrosis; viral infections such as cytomegalovirus and glandular fever; hemolytic anemias: this term includes the hereditary hemoglobinopathies such as thalassemia major and sickle cell anemia; the red cell fragility disorders, such as hereditary spherocytosis; and the red cell enzyme defects including glucose-6-phosphate dehydrogenase abnormalities; myeloproliferative disorders such as chronic myelogenous and chronic lymphatic leukemias; lymphoproliferative disorders such as the hairy cell lymphomas; chronic infections such as malaria, syphilis, kala-azar; acute infections such as bacterial endocarditis.

While splenomegaly may be often symptomless, it can also produce pain in the left hypochondrium, hiccups, feeling of fullness and loss of appetite, and early satiety after eating very little. Symptoms due to cytopenia may be present in cases of hypersplenism.

As stated above, splenomegaly is a condition which usually produces minimal symptoms, but it may be palpated when enlarged, as its lower border descends lower than the inferior edge of the ribcage in this situation. In such a case, further tests may be needed, such as imaging with ultrasound or CT scanning, and tests of peripheral blood to identify the kind and numbers of each type of cell which may give a clue to the etiology.

Treatment for splenomegaly is always specific to the underlying condition. In some cases, the spleen may have to be removed, but this is rare. In cases due to malignancy, chemotherapy or radiotherapy may be required. Blood transfusions may be necessary in patients with hemolytic anemia.

As for treatments of splenomegaly, one may cite, e.g., Okuyama et al. (Science Reports of the Research Institutes, 1983, Vol. 30(1-4), p. 29-30), which disclosed the therapeutic use of adrenaline in promoting an alleviation of splenomegaly, and Ferrannini et al. (Minerva Medica, 1947, Vol. 2(29), p. 45-51), which disclosed the therapeutic use of adrenaline in treating hepatic cirrhosis-induced splenomegaly. However, adrenaline binds to a great deal of receptors, including beta adrenergic receptors, and hence may interfere with various physiological processes.

Similarly, Neidhart (Experientia, 1996, Vol. 52(9), p. 892-899) reported that administration of 1 mg/kg of bromocriptine microcapsule (CBLA) reduced splenomegaly in a rat model for adjuvant arthritis, and U.S. Ser. No. 10/426,772 disclosed that ergotamine reduced flatworm-induced splenomegaly (schistosomiasis). However, both bromocriptine and ergotamine bind to several receptors and the therapeutic treatment cannot be specific, as it may affect other pathways.

CN106619876 and CN108174957 disclosed pharmaceutical compositions, respectively for treating anorexia and invigorating the spleen, and for tonifying the spleen.

Finally, WO2020050290 described that guanabenz has the capacity of promoting a decrease in the weight of the spleen in patient with obesity-related diseases, such as fatty liver.

There is therefore a need to provide the state of the art with alternative means to prevent and/or treat spleen disorders, in particular splenomegaly. There is a need to provide the state of the art with means to prevent and/or treat spleen disorders, in particular splenomegaly, irrespective of the underlying causing medical conditions.

There is also a need to provide the state of the art with alternatives to splenectomy, i.e. the removal of a diseased spleen.

### SUMMARY

A first aspect of the invention relates to an alpha-2 adrenergic receptor agonist for use in the prevention and/or the treatment of a spleen disorder.

In some embodiments, said spleen disorder is splenomegaly. In certain embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof. In certain embodiments, said agonist is selected in the group consisting of brimonidine, clonidine, dexmedetomidine, guanabenz, guanfacine, lofexidine, methyldopa, tizanidine, xylazine, and a functional derivative thereof, in particular clonidine, guanabenz, romifidine, or a functional derivative thereof. In some embodiments, the alpha-2 adrenergic receptor agonist is selected in the group consisting of an antibody, an antibody fragment, an afucosylated antibody, a diabody, a triabody, a tetrabody, a nanobody, and an analog thereof. In certain embodiments, said agonist does not cross the blood/brain barrier. In some embodiments, said agonist is to be administered at a dose ranging from about 0.0001 mg/kg body weight to about 100 mg/kg body weight. In some embodiments, said spleen disorder, in particular splenomegaly, is associated with a disorder selected in the group consisting of a hypersplenism, cancer, liver disorder, cystic fibrosis, an inflammatory disease, a microbial infection, a hemolytic anemia, and the like. In certain embodiments, said spleen disorder, in particular splenomegaly, is associated with blood cancer or solid cancer. In certain embodiments, said agonist is to be administered with a primary or a secondary treatment selected in the group consisting of antimicrobial agents, anti-inflammatory agents, chemotherapy, immunotherapy, radiation, the like, and a combination thereof.

In some embodiments, said immunotherapy comprises adoptive transfer of immune cells, a checkpoint inhibitor, vaccination, the like, and a combination thereof. In certain embodiments, said immune cells are selected in the group comprising T cells, in particular CD8+ T cells and CAR T cells; natural killer (NK) cells, in particular CAR NK cells; the like; and a combination thereof. In one embodiment, said primary or secondary treatment is to be administered separately or concomitantly with said agonist.

Another aspect of the invention also pertains to a pharmaceutical composition comprising an alpha-2 adrenergic receptor agonist, as defined in the instant invention, and a pharmaceutically acceptable vehicle, for use in the prevention and/or the treatment of a spleen disorder.

A further aspect of the invention relates to a combination kit comprising (i) an alpha-2 adrenergic receptor agonist or a pharmaceutical composition comprising an alpha-2 adrenergic receptor agonist and (ii) a therapeutic agent selected in the group consisting of an antibiotic, an antiparasitic, an antiviral, a chemotherapy agent, an immunotherapy agent, and the like, for use for the prevention and/or the treatment of a spleen disorder.

One aspect of the invention also relates to a method for the prevention and/or the treatment of a spleen disorder in an individual in need thereof comprising the administration of a therapeutically efficient amount of an alpha-2 adrenergic receptor agonist.

Another aspect of the invention relates to a method for reducing the volume and/or the weight of a spleen in an individual in need thereof comprising the administration of a therapeutically efficient amount of an alpha-2 adrenergic receptor agonist.

### Definitions

In the present invention, the following terms have the following meanings:
“About” preceding a figure encompasses plus or minus 10%, or less, of the value of said figure. It is to be understood that the value to which the term “about” refers to is itself also specifically, and preferably, disclosed.
“Comprise” is intended to mean “contain”, “encompass” and “include”. In some embodiments, the term “comprise” also encompasses the term “consist of”.
“Spleen disorder” refers to a disorder that involves an abnormal shape, size, weight or volume, and/or an abnormal functioning of the spleen. As used herein, the spleen disorder may be diagnosed by skilled authorized personnel, by any suitable means usually employed in the field. Illustratively, blood tests which allow assessing the complete blood cells count; ultrasound, computerized tomography (CT) or magnetic resonance imaging (MRI) scan, which allow assessing the size of the spleen and whether it's crowding other organs, may provide valuable information about spleen disorders.
“Splenomegaly” refers to an abnormal enlargement of the spleen. In other words, splenomegaly concerns an increase of the volume and/or the weight of the spleen, as compared to a healthy spleen. Splenomegaly is often a sign of an underlying condition, such as, e.g., severe liver disease, leukemia, or mononucleosis.
“Alpha-2 adrenergic receptor agonist” refers to a compound capable of activating alpha-2 adrenergic receptors, upon binding to those receptors. Within the scope of the instant invention, the term “activating alpha-2 adrenergic receptors” is intended to mean that upon activation, these receptors are capable of inhibiting norepinephrine release from presynaptic neurons, and/or centrally inducing sedation via locus coeruleus, and/or inhibiting insulin release from pancreatic 13 cells.
“Functional derivative”, when referred to the alpha-2 adrenergic receptor agonist according to the invention, is intended to refer to a derivate of an alpha-2 adrenergic receptor agonist that shares an equivalent biological physiological function, while having a similar structure. The term “having a similar structure” is intended to mean that the derivate of the alpha-2 adrenergic receptor agonist differs from the reference alpha-2 adrenergic receptor agonist in that it possesses one or more substituent(s). As used herein, the term “substituents” include linear or ramified groups, in particular alkyl, aryl, amine, amide, sulfide, bromide, chloride and/or fluoride groups. Within the scope of the invention, an alpha-2 adrenergic receptor agonist functional derivative is an agonist of the alpha-2 receptor.
“Treating” or “treatment” or “alleviation” refers to both therapeutic treatment and prophylactic or preventative measures, wherein the object is to prevent or slow down (lessen) the targeted pathologic spleen condition or disorder, in particular a splenomegaly. Those in need of treatment include those already with said disorder as well as those prone to develop the disorder or those in whom the disorder is to be prevented. An individual is successfully “treated” for a spleen disorder, in particular splenomegaly, if, after receiving a therapeutic amount of an alpha-2 adrenergic receptor agonist according to the present invention, the individual shows observable and/or measurable reduction in, or absence of, one or more of the symptoms associated with said spleen disorder, in particular splenomegaly; reduced morbidity and mortality, and improvement in quality of life issues. The above parameters for assessing successful treatment and improvement in the disease are readily measurable by routine procedures familiar to physician or authorized personnel.
“Preventing” refers to keeping from happening, and/or lowering the chance of the onset of, or at least one adverse effect or symptom of, a spleen disorder, in particular splenomegaly.
“Therapeutically efficient amount” refers to the level or the amount of the active agent that is aimed at, without causing significant negative or adverse side effects to the target, (1) delaying or preventing the onset of a spleen disorder, in particular splenomegaly; (2) slowing down or stopping the progression, aggravation, or deterioration of one or more symptoms of a spleen disorder, in particular splenomegaly; (3) bringing about ameliorations of the symptoms of a spleen disorder, in particular splenomegaly; (4) reducing the severity or incidence of a spleen disorder, in particular splenomegaly; or (5) curing a spleen disorder, in particular splenomegaly. A therapeutically effective amount may be administered prior to the onset of a spleen disorder, in particular splenomegaly, for a prophylactic or preventive action. Alternatively, or additionally, the therapeutically effective amount may be administered after the onset of a spleen disorder, in particular splenomegaly, for a therapeutic action. In one embodiment, a therapeutically effective amount of the composition is an amount that is effective in reducing at least one symptom of a spleen disorder, in particular splenomegaly.
“Pharmaceutically acceptable vehicle” refers to a vehicle that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the FDA (Food and Drug Administration) in the United States or the EMA (European Medicines Agency) in the European Union.
“Individual” is intended to refer to an animal individual, preferably a mammalian individual, more preferably a human individual. Among the non-human mammalian individuals of interest, one may non-limitatively mention pets, such as dogs, cats, guinea pigs; animals of economic importance such as cattle, sheep, goats, horses, monkeys. In one embodiment, an individual may be a “patient”, i.e. a warm-blooded animal, more preferably a human, who/which is awaiting the receipt of, or is receiving medical care or was/is/will be the object of a medical procedure, or is monitored for the development of a spleen disease, disorder or condition. In one embodiment, the individual is an adult (for example a human subject above the age of 18). In another embodiment, the individual is a child (for example a human subject below the age of 18). In one embodiment, the individual is a male. In another embodiment, the individual is a female.

### DETAILED DESCRIPTION

By the means of in vivo experimental data obtained with several models of induced splenomegaly, the inventors surprisingly showed herein that alpha-2 adrenergic receptor agonists can be used as a monotherapy for the treatment of spleen disorders. More particularly, it is the agonistic mechanism that is important to observe the therapeutic effect towards the spleen, as demonstrated by the absence of any therapeutic properties in the presence of an antagonist of the alpha-2 adrenergic receptor or in an alpha-2 adrenergic receptor knock-out mice. The inventors hence demonstrated that the structure of the alpha-2 adrenergic receptor agonists is not at stake to explain the benefit towards spleen disorders' treatment but rather their function. In addition, the inventors herein provided evidences that the beneficial properties of alpha-2 adrenergic receptor agonists towards the spleen's health are applicable to various types of spleen disorders, such as cancer-induced splenomegaly, splenomegaly induced by virus-mediated or protein-mediated immunization, or splenomegaly induced by myelofibrosis.

In one aspect, the invention relates to an alpha-2 adrenergic receptor agonist for use in the prevention and/or the treatment of spleen disorder. Another aspect of the invention relates to an alpha-2 adrenergic receptor agonist for use in the treatment of spleen disorder.

In practice, a spleen disorder, in particular splenomegaly, may be diagnosed by any suitable method known in the state of the art, or method adapted therefrom. Non-limitative examples of suitable methods include blood tests, such as a complete blood count (numbers of red blood cells, white blood cells and platelets); ultrasound or computerized tomography (CT) scan, so as to determine the size of the spleen and to evaluate whether the spleen is crowding other organs; magnetic resonance imagining (MRI), so as to trace blood flow through the spleen.

In practice, the alpha-2 adrenergic receptor agonist is the active agent for use in the prevention and/or the treatment of spleen disorder. In some embodiments, the alpha-2 adrenergic receptor agonist is the sole active agent for use in the prevention and/or the treatment of spleen disorder. In certain embodiments, the alpha-2 adrenergic receptor agonist is to be administered as a monotherapy for the prevention and/or the treatment of spleen disorder. In some embodiments, the alpha-2 adrenergic receptor agonist is to be administered as a monotherapy for the treatment of spleen disorder.

As used herein, the term “monotherapy” is intended to mean that the alpha-2 adrenergic receptor agonist according to the invention representing the sole therapeutic compound for the use in the prevention and/or treatment of spleen disorder.

In certain embodiments, the spleen disorder is splenomegaly.

In some embodiments, the alpha-2 adrenergic receptor agonist is a small organic molecule, a peptide, a polypeptide or a protein.

As used herein, the term “small organic molecule” is intended to refer to an organic molecule that has a molar weight inferior to about 1,000 g/mol, preferably inferior to about 750 g/mol, more preferably inferior to about 600 g/mol. Within the scope of the invention, the expression “inferior about 1,000 g/mol” encompasses inferior to about 1,000 g/mol, 950 g/mol, 900 g/mol, 850 g/mol, 800 g/mol, 750 g/mol, 700 g/mol, 650 g/mol, 600 g/mol, 550 g/mol, 500 g/mol, 450 g/mol, 400 g/mol, 350 g/mol, 300 g/mol, 250 g/mol and 200 g/mol. In practice, the molar weight of a molecule may be determined by any suitable methods acknowledged in the state of the art, or a method adapted therefrom. Non-limitative examples of suitable methods include mass spectrometry, nuclear magnetic resonance (NMR), and the like.

As used herein, the term “peptide” refers to a linear polymer of amino acids of less than 50 amino acid residues linked together by peptide bonds; the term “polypeptide” refers to a linear polymer of at least 50 amino acid residues linked together by peptide bonds; and the term “protein” specifically refers to a functional entity formed of one or more peptides or polypeptides

In certain embodiments, the alpha-2 adrenergic receptor agonist is a small organic molecule.

In some embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

As defined herein, it is to be understood that amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R) nitrobiphenyline, synephrine, talipexole, tizanidine, xylazine, xylometazoline, and a functional derivative thereof, are small organic molecules.

In certain embodiments, said agonist is selected in the group consisting of brimonidine, clonidine, dexmedetomidine, guanabenz, guanfacine, lofexidine, methyldopa, romifidine, tizanidine, xylazine, and a functional derivative thereof. In certain embodiments, said agonist is selected in the group consisting of brimonidine, clonidine, guanfacine, lofexidine, methyldopa, romifidine, tizanidine, and a functional derivative thereof. In particular, in certain embodiments, said agonist is clonidine, guanabenz or romifidine, or a functional derivative thereof.

In some embodiments, the alpha-2 adrenergic receptor agonist is not guanabenz, or a functional derivative thereof. In certain embodiments, the alpha-2 adrenergic receptor agonist is not bromocriptine, or a functional derivative thereof. In some embodiments, the alpha-2 adrenergic receptor agonist is not ergotamine, or a functional derivative thereof. In certain embodiments, the alpha-2 adrenergic receptor agonist is not dexmedetomidine, or a functional derivative thereof. In some embodiments, the alpha-2 adrenergic receptor agonist is not xylazine, or a functional derivative thereof. In certain embodiments, the alpha-2 adrenergic receptor agonist is not dl-methylephedrine, or a functional derivative thereof. In certain embodiments, the alpha-2 adrenergic receptor agonist is not synephrine, or a functional derivative thereof.

In some embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanfacine, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof. In certain embodiments, said agonist is selected in the group consisting of brimonidine, clonidine, dexmedetomidine, guanfacine, lofexidine, methyldopa, tizanidine, xylazine, and a functional derivative thereof.

In certain embodiments, said agonist is selected in the group consisting of amitraz, apraclonidine, bethanidine, brimonidine, cirazoline, clonidine, detomidine, dipivefrin, droxidopa, epinephrine, etilefrine, etomidate, fadolmidine, guanfacine, guanethidine, indanidine, lofexidine, medetomidine, mephentermine, metamfetamine, metaraminol, methoxamine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, talipexole, tizanidine, xylometazoline, and a functional derivative thereof.

In one particular embodiment, said agonist is clonidine or romifidine, or a functional derivative thereof. In one particular embodiment, said agonist is clonidine or a functional derivative thereof.

In certain embodiments, the alpha-2 adrenergic receptor agonist is a peptide, a polypeptide or a protein.

In some embodiments, the alpha-2 adrenergic receptor agonist is selected in the group consisting of an antibody, an antibody fragment, an afucosylated antibody, a diabody, a triabody, a tetrabody, a nanobody, and an analog thereof.

As used herein, an “antibody”, also referred to as immunoglobulins (abbreviated “Ig”), is intended to refer to a gamma globulin protein that is found in blood or other bodily fluids of vertebrates, and is often used by the immune system to identify and neutralize foreign objects, such as bacteria and viruses. Antibodies consist of two pairs of polypeptide chains, called heavy chains and light chains that are arranged in a Y-shape. The two tips of the Y are the regions that bind to antigens and deactivate them. The term “antibody” (Ab) as used herein includes monoclonal antibodies, polyclonal antibodies, multi-specific antibodies (e.g., bispecific antibodies), and antibody fragments, so long as they exhibit the desired biological activity. The term “immunoglobulin” (Ig) is used interchangeably with “antibody” herein.

As used herein, an “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 1995; 8(10): 1057-1062); single-chain antibody molecules; and multi-specific antibodies formed from antibody fragments. One may refer to a “functional fragment or analog” of an antibody, which is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc[epsilon]RI. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

As used herein, an “afucosylated antibody” refers to an antibody lacking core fucosylation. As a matter of fact, nearly all IgG-type antibodies are N-glycosylated in their Fc moiety. Typically, a fucose residue is attached to the first N-acetylglucosamine of these complex-type N-glycans. In other words, an “afucosylated antibody” refers to an antibody that does not possess N-glycans.

As used herein, the term “diabody” refers to a small antibody fragment prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described in more details in, e.g., EP0404097; WO 93/11161; and Hollinger et al., Proc. Natl. Acad. Sci. USA, 1993; 90:6444-6448.

As used herein, a “triabody” is intended to refer to an antibody that has three Fv heads, each consisting of a VH domain from one polypeptide paired with the VL domain from a neighboring polypeptide.

As used herein, a “nanobody” refers to a functional antibody that consists of heavy chains only and therefore lack light chains. These heavy-chain only antibodies contain a single variable domain (VHH) and two constant domains (CH2, CH3).

In some embodiments, the alpha-2 adrenergic receptor agonist according to the invention is not an agonist of the alpha-1 adrenergic receptor. In certain embodiments, the alpha-2 adrenergic receptor agonist according to the invention is not an agonist of one beta adrenergic receptor, in particular the beta-1 adrenergic receptor, the beta-2 adrenergic receptor and/or the beta-3 adrenergic receptor.

In certain embodiments, said agonist does not cross the blood/brain barrier.

In some embodiments, the agonist that does not cross the blood/brain barrier is selected in the group consisting of apraclonidine, ST-91 (also referred to as N-(2,6-Diethylphenyl)-4,5-dihydro-1H-imidazol-2-amine hydrochloride), corbadrine (also referred to as 4-(2-amino-1-hydroxy-propyl)benzene-1,2-diol, or α-methyl-norepinephrine), and naphazoline.

In practice, assessing whether a compound, e.g., an alpha-2 adrenergic receptor agonist according to the invention, crosses the blood/brain barrier may be performed by any suitable method acknowledged from the state of the art, or a method adapted therefrom. Illustratively, this assessment may be performed by the means of one of the two gold-standard experimental measures of blood/brain barrier permeability, namely, (1) log BB, which is intended to measure the concentration of a compound in the brain divided by concentration in the blood; and (2) log PS, which measures the permeability surface-area product.

In certain embodiments, alpha-2 adrenergic receptor agonists according to the invention also encompass functional derivatives of alpha-2 adrenergic receptor agonists.

As used herein, the term “functional derivative”, when referred to an alpha-2 adrenergic receptor agonist according to the invention, is intended to refer to a compound that is both structurally and functionally related to a reference alpha-2 adrenergic receptor agonist, i.e., an agonist for which experimental data exist showing that said agonist binds specifically to the alpha-2 adrenergic receptor.

In practice, the functional derivative of an agonist according to the invention is an agonist that bears one or more substituent(s), in particular selected from the group consisting of an alkyl group, an aryl group, an amine group, an amide group, a sulfide group, a bromide group, a chloride group, a fluoride group, carbonyl (C═O) group, formyl group, hydroxyl group, aldehyde group, alkoxy (O—R) group. Other groups further include: alkenyl, alkynyl, carboxamide, primary amine, R₂NH, R₃N, R₄N⁺, azide, azo(diimide), benzyl, carbonate ester, carboxylate, carboxyl, cyanate, RSCN, disulfide, ether, ester, hydroperoxy, primary ketimine, RC(═NR)R′, RC(═NH)H, RC(═NR′)H, imide, isocyanide, isocyanate, RNCS, nitrate, nitrile, nitrosooxy, nitro, nitroso, peroxy, phenyl, phosphino, phosphate, phosphono, pyridyl, sulfonyl, sulfo, sulfinyl, sulfhydryl, fluoro, chloro, bromo, iodo, haloformyl, carboalkoxy, hemiacetal, hemiketal, acetal, ketal, orthoester, methylenedioxy, orthocarbonate ester, carboxylic anhydride, imine, azo(diimide), isonitrile, oxime, carbamate, sulfide, sulfino, thiocyanate, isothiocyanate, carbonothioyl, carbothioic o-acid, thiolester, thionoester, carbodithioic acid, carbodithio, borono, boronate, borino, borinate, alkyllithium, alkylmagnesium halide, alkylaluminium, silyl ether group, and any combination thereof. In practice, R and R′ groups are non-limitatively referring to alkyl, alkenyl, alkynyl groups. It is to be understood that functional derivative possesses itself the capacity of activating the alpha-2 adrenergic receptor, hence is an alpha-2 adrenergic receptor agonist.

In some embodiments, the spleen disorder is a mammalian spleen disorder, in particular a non-human spleen disorder. In some embodiments, the spleen disorder in a human spleen disorder. In one embodiment, the spleen disorder in human splenomegaly.

In certain embodiments, said spleen disorder, in particular splenomegaly, is associated with a disorder selected in the group consisting of a hypersplenism, cancer, liver disorder, cystic fibrosis, an inflammatory disease, a microbial infection, a hemolytic anemia, and the like.

In some embodiments, said spleen disorder, in particular splenomegaly, is associated with a blood cancer or a solid cancer.

As used herein, the term “blood cancer”, also referred to as “hematologic cancer”, encompasses any cancer involving uncontrolled proliferation of blood cells, in particular white blood cells. Blood cancers includes bone marrow cancer, such as myelofibrosis; leukemia; lymphoma (Hodgkin and non-Hodgkin lymphomas); and myeloma.

In certain embodiments, said cancer is a blood cancer selected in the group consisting of myelofibrosis, Hodgkin's disease, immunoblastic lymphadenopathy, lymphoma, chronic lymphocytic leukemia, acute leukemia, and the like. In some embodiments, the blood cancer is myelofibrosis.

As used herein, the term “solid cancer” encompasses any cancer (also referred to as malignancy) that forms a discrete tumor mass, as opposed to cancers (or malignancies) that diffusely infiltrate a tissue without forming a mass.

In some embodiments, said solid cancer is selected from the group consisting of adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, kidney cancer, lung cancer, melanoma, Merkel cell skin cancer, mesothelioma, multiple myeloma, myeloproliferative disorders, ovarian cancer, pancreatic cancer, prostate cancer, salivary gland cancer, sarcoma, squamous cell carcinoma, testicular cancer, thyroid cancer, urothelial carcinoma, uveal melanoma, and the like. In certain embodiments, the solid cancer is selected from the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma, pancreatic carcinoma, and the like.

In some embodiments, said solid cancer is selected in the group consisting of carcinoma, melanoma, ovarian cancer, and the like.

In certain embodiments, said inflammatory disease is selected in the group consisting of rheumatoid arthritis, lupus, sarcoidosis, and the like. In one embodiment, the spleen disorder, in particular splenomegaly, is not hepatic cirrhosis-induced splenomegaly. In one embodiment, the spleen disorder, in particular splenomegaly, is not arthritis-induced splenomegaly.

In some embodiments, said microbial infection is selected in the group consisting of malaria, infectious mononucleosis, and the like. In one embodiment, the spleen disorder, in particular splenomegaly, is not a worm-induced splenomegaly, preferably is not schistosomiasis-induced splenomegaly.

In some embodiments, said spleen disorder, in particular splenomegaly, is associated with a disorder selected in the group consisting of spherocytosis, amyloidosis, sickle cell disease, idiopathic thrombocytopenic purpura, hemoglobinopathies, Gaucher and Niemann-Pick diseases, histiocytosis, and the like. In one embodiment, the spleen disorder, in particular splenomegaly, is not associated with obesity or an obesity-related disease. In one embodiment, the spleen disorder, in particular splenomegaly, is not associated with fatty liver. In one embodiment, the spleen disorder, in particular splenomegaly, is not associated with metabolic syndrome.

It is to be understood that the therapeutic use of the alpha-2 adrenergic receptor agonist according to the instant invention, which does not include the prevention of a spleen disorder, is not intended to be of use for invigorating and/or tonifying the spleen. As used herein, the term “invigorating and/or tonifying the spleen” refers to the action of maintaining or stimulating the physiological function of a healthy, non-diseased spleen.

In practice, the total daily dose of the agonist according to the invention may be decided by the attending physician within the scope of sound medical judgment. The specific dose for any particular subject will depend upon a variety of factors such as the severity of the spleen disorder, in particular the splenomegaly, to be treated. Illustratively, these factors include age, body weight, general health, gender and diet of the patient, and additional factors well-known in the medical arts.

In certain embodiments, said agonist is to be administered at a dose ranging from about 0.0001 mg/kg body weight to about 100 mg/kg body weight.

Within the scope of the instant invention, the term “about 0.0001 mg/kg body weight to about 100 mg/kg body weight” encompasses about 0.0001, 0.0002, 0.0003, 0.0004, 0.0005, 0.0006, 0.0007, 0.0008, 0.0009, 0.001, 0.002, 0.0003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95 and 100 mg/kg body weight.

In certain embodiments, the dose is ranging from about 0.0001 mg/kg body weight to about 30 mg/kg body weight, preferably from about 0.0001 mg/kg body weight to about 15 mg/kg body weight, more preferably from about 0.0001 mg/kg body weight to about 7 mg/kg body weight. In some embodiments, the dose is ranging from about 0.01 mg/kg body weight to about 30 mg/kg body weight, preferably from about 0.01 mg/kg body weight to about 15 mg/kg body weight, more preferably from about 0.01 mg/kg body weight to about 7 mg/kg body weight.

In certain embodiments, the daily dose is ranging from about 0.0001 mg/day/kg body weight to about 100 mg/day/kg body weight, in particular from about 0.001 mg/day/kg body weight to about 30 mg/day/kg body weight, preferably from about 0.001 mg/day/kg body weight to about 15 mg/day/kg body weight, more preferably from about 0.001 mg/day/kg body weight to about 7 mg/day/kg body weight. In certain embodiments, the daily dose is ranging from about 0.01 mg/kg/day body weight to about 100 mg/kg/day body weight, in particular from about 0.01 mg/kg/day body weight to about 30 mg/kg/day body weight, preferably from about 0.01 mg/kg/day body weight to about 15 mg/kg/day body weight, more preferably from about 0.01 mg/kg/day body weight to about 7 mg/kg/day body weight.

In certain embodiments, the agonist according to the invention may be, or is to be, administered orally, systemically, parenterally, topically, by inhalation spray, rectally, nasally, buccally, vaginally or via an implanted medical device. In some embodiments, the alpha-2 adrenergic receptor agonist according to the invention may be, or is to be, administered systemically.

According to some embodiments, the agonist according to the instant invention is formulated in a suitable form for an oral administration. Thus, in one embodiment, said agonist is to be administered orally to the subject, for example in the form of a powder, a tablet, a capsule, and the like or as a tablet formulated for extended or sustained release. Non-limitative examples of forms suitable for oral administration include, e.g., liquid, paste or solid compositions, and more particularly tablets, tablets formulated for extended or sustained release, capsules, pills, dragees, liquids, gels, syrups, slurries, and suspensions.

According to another embodiment, the agonist according to the invention is in an adapted form for an injection. Illustratively, said agonist is thus to be injected to the subject, by intravenous, intramuscular, intraperitoneal, intrapleural, subcutaneous, transdermal injection or infusion. Sterile injectable forms of the agonist according to the invention may include solutions, aqueous or oleaginous suspensions. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to another embodiment, the agonist according to the invention is in an adapted form for a topical administration. Examples of forms adapted for topical administration include, without being limited to, liquid, paste or solid compositions, and more particularly aqueous solutions, drops, dispersions, sprays, microcapsules, nanoparticles, microparticles, polymeric patch, or controlled-release patch, and the like.

In some embodiments, said agonist is to be administered with a primary or a secondary treatment selected in the group consisting of antimicrobial agents, anti-inflammatory agents, chemotherapy, immunotherapy, radiation, the like, and a combination thereof.

As used herein, antimicrobial agents include antibiotics, antiparasitics, antivirals.

In some embodiments, the antimicrobial agent is an antibiotic, in particular selected in the group consisting of a penicillin, such as, e.g., ampicillin, amoxicillin and dicloxacillin; a tetracycline such as, e.g., demeclocycline, doxycycline, eravacycline, minocycline, omadacycline and tetracycline; a cephalosporin, such as, e.g., cefaclor, cefdinir, cefotaxime, ceftazidime, ceftriaxone, and cefuroxime; a quinolone such as, e.g., ciprofloxacin, levofloxacin and moxifloxacin; a lincomycin, such as, e.g., clindamycin and lincomycin; a macrolide such as, e.g., azithromycin, clarithromycin and erythromycin; a sulfonamide such as, e.g., sulfasalazine, sulfamethoxazole and trimethoprim; a glycopeptide such as, e.g., dalbavancin, oritavancin, telavancin and vancomycin; an aminoglycoside such as, e.g., amikacin, gentamicin and tobramycin; a carbapenem such as, e.g., doripenem, ertapenem, meropenem, and the like.

In some embodiments, the antimicrobial agent is an antiparasitic, in particular selected in the group consisting of nitazoxanide, melarsoprol, atovaquone, proguanil, quinine, artesunate, artemisinin, eflornithine, metronidazole, tinidazole, miltefosine, mebendazole, pyrantel pamoate, thiabendazole, diethylcarbamazine, ivermectin, niclosamide, praziquantel, albendazole, praziquantel, rifampin, amphotericin B, fumagillin, and the like.

In some embodiments, the antimicrobial agent is an antiviral, in particular selected in the group consisting of abacavir, acyclovir, adefovir, amantadine, ampligen, amprenavir, arbidol, atazanavir, atripla, balavir, baloxavir marboxil, biktarvy, boceprevir, cidofovir, cobicistat, combivir, daclatasvir, darunavir, delavirdine, descovy, didanosine, docosanol, dolutegravir, doravirine, ecoliever, edoxudine, efavirenz, elvitegravir, emtricitabine, enfuvirtide, entecavir, etravirine, famciclovir, fomivirsen, fosamprenavir, foscarnet, fosfonet, ganciclovir, ibacitabine, ibalizumab, idoxuridine, imiquimod, imunovir, indinavir, inosine, interferon type I, interferon type II, interferon type III, lamivudine, letermovir, lopinavir, loviride, maraviroc, methisazone, moroxydine, nelfinavir, nevirapine, nexavir, nitazoxanide, norvirn, oseltamivir, peginterferon alfa-2a, peginterferon alfa-2b, penciclovir, peramivir, pleconaril, podophyllotoxin, pyramidine, raltegravir, remdesivir, ribavirin, rilpivirine, rimantadine, ritonavir, saquinavir, simeprevir, sofosbuvir, stavudine, telaprevir, telbivudine, tenofovir alafenamide, tenofovir disoproxil, tenofovir, tipranavir, trifluridine, trizivir, tromantadine, Truvada, valaciclovir, valganciclovir, vicriviroc, vidarabine, viramidine, zalcitabine, zanamivir, zidovudine, and the like.

Non-limitative examples of anti-inflammatory agents include aspirin, celecoxib, diclofenac, etoricoxib, ibuprofen, indomethacin, mefenamic acid, naproxen, oxaprozin, piroxicam, and the like.

As used herein, the term “chemotherapy” refers to a drug treatment that uses chemicals to kill fast-growing cells, in particular cancer cells.

Non-limitative examples of chemotherapy agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the like.

As used herein, the term “immunotherapy” refers to a therapy aiming at inducing and/or enhancing an immune response towards a specific target, for example towards infectious agents such as viruses, bacteria, fungi or protozoan parasites, or towards cancer cells. As used herein, examples of immunotherapies include, without being limited to, vaccination, such as preventive and therapeutic vaccination; adoptive transfer of immune cells, in particular of T cells (such as alpha beta (4) T cells or gamma delta T cells) or NK cells; checkpoint inhibitors; checkpoint agonists; antibodies.

In some embodiments, the immunotherapy is a cancer immunotherapy. As used herein, the term “cancer immunotherapy” refers to an immunotherapy used for the treatment of a cancer, said immunotherapy modulating the immune response of a subject with the aim of inducing and/or stimulating the immune response of the subject towards cancer cells. In one embodiment, the cancer immunotherapy comprises or consists of the adoptive transfer of immune cells, in particular of T cells (such as alpha beta (4) T cells or gamma delta T cells), NK cells or NK T cells. In one embodiment, the cancer immunotherapy comprises or consists of the administration of a checkpoint inhibitor. In one embodiment, the cancer immunotherapy comprises or consists of the administration of a checkpoint agonist. In one embodiment, the cancer immunotherapy comprises or consists of the administration of an antibody. In one embodiment, the cancer immunotherapy comprises or consists of the administration of a therapeutic anti-cancer vaccine.

In certain embodiments, said immunotherapy comprises adoptive transfer of immune cells, a checkpoint inhibitor, vaccination, the like, and a combination thereof.

As used herein, an adoptive transfer of cells or adoptive cell therapy (or ACT) is defined as the transfer, for example as an infusion, of immune cells to a subject. As a cancer treatment, the adoptive transfer of immune cells to a subject aims at enhancing the subject immune response towards the cancer cells.

In one embodiment, the transferred immune cells are T cells or natural killer (NK) cells. In one embodiment, the transferred immune cells are T cells, in particular CD8+ T cells, and/or natural killer (NK) cells.

In one embodiment, the transferred immune cells are cytotoxic cells. Examples of cytotoxic cells include natural killer (NK) cells, CD8+ T cells, and natural killer (NK) T cells.

In one embodiment, the transferred immune cells are natural killer (NK) cells.

In one embodiment, the transferred immune cells are T cells, in particular effector T cells. Examples of effector T cells include CD4+ T cells and CD8+ T cells. In one embodiment, the transferred immune cells are alpha beta (αβ) T cells. In another embodiment, the transferred immune cells are gamma delta (γδ) T cells. In one embodiment, the transferred immune cells are CD4+ T cells, CD8+ T cells, or natural killer (NK) T cells, preferably the transferred T cells are CD8+ T cells.

In one embodiment, the transferred immune cells as described hereinabove are antigen-specific immune cells. In one embodiment, the transferred immune cells as described hereinabove are antigen-specific immune cells, wherein said antigen is specifically and/or abundantly expressed by cancer cells. In one embodiment, the transferred immune cells as described hereinabove are tumor-specific immune cells, in other words the transferred immune cells as described hereinabove specifically recognize cancer cells or tumor cells through an antigen specifically and/or abundantly expressed by said cancer cells or tumor cells. In one embodiment, the transferred immune cells as described hereinabove are tumor-specific effector T cells. In one embodiment, the transferred immune cells as described hereinabove are tumor-specific CD8+ effector T cells, in particular tumor-specific cytotoxic CD8+ T cells. In one embodiment, the transferred immune cells as described hereinabove are tumor-specific cytotoxic cells.

In one embodiment, the transferred immune cells as described hereinabove are tumor-specific NK cells.

Examples of tumor-specific antigens, i.e., antigens that are specifically and/or abundantly expressed by cancer cells include, without being limited to, neoantigens (also referred to as new antigens or mutated antigens), 9D7, ART4, β-catenin, BING-4, Bcr-abl, BRCA1/2, calcium-activated chloride channel 2, CDK4, CEA (carcinoembryonic antigen), CML66, Cyclin B1, CypB, EBV (Epstein-Barr virus) associated antigens (such as LMP-1, LMP-2, EBNA1 and BARF1), EGFRvIII, Ep-CAM, EphA3, fibronectin, Gp100/pmel17, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-2, p53, PRAME, prostate-specific antigen (PSA), PSMA (prostate-specific membrane antigen), Ras, SAP-1, SART-I, SART-2, SART-3, SSX-2, survivin, TAG-72, telomerase, TRP-1/-2, tyrosinase, WT1, antigens of the BAGE family, antigens of the CAGE family, antigens of the GAGE family, antigens of the MAGE family, antigens of the SAGE family, and antigens of the XAGE family.

As used herein, neoantigens (also referred to as new antigens or mutated antigens) correspond to antigens derived from proteins that are affected by somatic mutations or gene rearrangements acquired by the tumors. Neoantigens may be specific to each individual subject and thus provide targets for developing personalized immunotherapies.

Examples of neoantigens include for example, without being limited to, the R24C mutant of CDK4, the R24L mutant of CDK4, KRAS mutated at codon 12, mutated p53, the V600E mutant of BRAF and the R132H mutant of IDH1.

In one embodiment, the transferred immune cells as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs corresponds to antigens encoded by genes that are expressed in tumor cells but not in normal tissues except in male germline cells.

Examples of CTAs include, without being limited to, MAGE-A1, MAGE-A3, MAGE-A4, MAGE-C2, NY-ESO-1, PRAME and SSX-2.

As used herein, the class of viral antigens corresponds to antigens derived from viral oncogenic proteins. Examples of viral antigens include, without being limited to, HPV (human papillomavirus) associated antigens such as E6 and E7, and EBV

(Epstein-Barr virus) associated antigens such as LMP-1, LMP-2, EBNA1 and BARF1.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

For example, autologous T cells can be generated ex vivo either by expansion of antigen-specific T cells isolated from the subject or by redirection of T cells of the subject through genetic engineering.

In one embodiment, the immune cells to be infused are modified ex vivo, in particular with RNA interference (also known as RNAi), before being infused to the subject.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In some embodiments, said immune cells are selected in the group comprising T cells, in particular CD8+ T cells and CAR T cells; natural killer (NK) cells, in particular CAR NK cells; the like; and a combination thereof.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

Examples of tumor-specific antigens are mentioned hereinabove.

In one embodiment, the transferred CAR T cells or CAR NK cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, EphA2, CD19, CD21, and CD20.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

As used herein, a checkpoint inhibitor therapy is defined as the administration of at least one checkpoint inhibitor to the subject.

Checkpoint inhibitors (CPI, that may also be referred to as immune checkpoint inhibitors or ICI) block the interactions between inhibitory receptors expressed on T cells and their ligands. As a cancer treatment, checkpoint inhibitor therapy aims at preventing the activation of inhibitory receptors expressed on T cells by ligands expressed by the tumor cells. Checkpoint inhibitor therapy thus aims at preventing the inhibition of T cells present in the tumor, i.e., tumor infiltrating T cells, and thus at enhancing the subject immune response towards the tumor cells.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of IDO (indoleamine 2,3-dioxygenase) and inhibitors of TDO (tryptophan 2,3-dioxygenase); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

In one embodiment, the at least one checkpoint inhibitor is selected from the group comprising or consisting of inhibitors or PD-1, inhibitors of PD-L1, inhibitors of CTLA 4 and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, avelumab, atezolizumab, durvalumab, LY3300054, ipilimumab, tremelimumab, and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, avelumab, atezolizumab, durvalumab, ipilimumab, tremelimumab, and any mixtures thereof.

In one embodiment, the at least one checkpoint inhibitor is an inhibitor of PD-1, also referred to as an anti-PD-1. Inhibitors of PD-1 may include antibodies targeting PD-1, in particular monoclonal antibodies, and non-antibody inhibitors such as small molecule inhibitors. Examples of inhibitors of PD-1 include, without being limited to, pembrolizumab, nivolumab, cemiplimab, tislelizumab, spartalizumab, ABBV-181, JNJ-63723283, BI 754091, MAG012, TSR-042, and AGEN2034. Pembrolizumab is also known as MK-3475, MK03475, lambrolizumab, or SCH-900475. The trade name of pembrolizumab is Keytruda®. Nivolumab is also known as ONO-4538, BMS-936558, MDX1106, or GTPL7335. The trade name of nivolumab is Opdivo®. Cemiplimab is also known as REGN2810 or REGN-2810. Tislelizumab is also known as BGB-A317. Spartalizumab is also known as PDR001 or PDR-001. In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, and any mixtures thereof.

In one embodiment, the at least one checkpoint inhibitor is an inhibitor of PD-L1, also referred to as an anti-PD-L1. Inhibitors of PD-L1 may include antibodies targeting PD-L1, in particular monoclonal antibodies, and non-antibody inhibitors such as small molecule inhibitors. Examples of inhibitors of PD-L1 include, without being limited to, avelumab, atezolizumab, durvalumab and LY3300054. Avelumab is also known as MSB0010718C, MSB-0010718C, MSB0010682, or MSB-0010682. The trade name of avelumab is Bavencio®. Atezolizumab is also known as MPDL3280A (clone YW243.55.S70), MPDL-3280A, RG-7446 or RG7446. The trade name of atezolizumab is Tecentriq®. Durvalumab is also known as MEDI4736 or MEDI-4736. The trade name of durvalumab is Imfinzi® In one embodiment, the at least one checkpoint inhibitor is selected from the group comprising or consisting of avelumab, atezolizumab, durvalumab, LY3300054, and any mixtures thereof. In one embodiment, the at least one checkpoint inhibitor is selected from the group comprising or consisting of avelumab, atezolizumab, durvalumab, and any mixtures thereof.

In one embodiment, the at least one checkpoint inhibitor is an inhibitor of CTLA-4, also referred to as an anti-CTLA-4. Inhibitors of CTLA-4 may include antibodies targeting CTLA-4, in particular monoclonal antibodies, and non-antibody inhibitors such as small molecule inhibitors. Examples of inhibitors of CTLA-4 include, without being limited to, ipilimumab and tremelimumab. Ipilimumab is also known as BMS-734016, MDX-010, or MDX-101. The trade name of ipilimumab is Yervoy®. Tremelimumab is also known as ticilimumab, CP-675, or CP-675,206. In one embodiment, the at least one checkpoint inhibitor is selected from the group comprising or consisting of ipilimumab, tremelimumab, and any mixtures thereof.

In one embodiment, the at least one checkpoint inhibitor is an inhibitor of IDO or an inhibitor of TDO, also referred to as an anti-IDO or anti-TDO, respectively. Examples of inhibitors of IDO include, without being limited to, 1-methyl-D-tryptophan (also known as indoximod), epacadostat (also known as INCB24360), navoximod (also known as IDO-IN-7 or GDC-0919), linrodostat (also known as BMS-986205), PF-06840003 (also known as E0S200271), TPST-8844, and LY3381916.

As used herein, an antibody therapy is defined as the administration of at least one antibody to the subject.

As a cancer treatment, antibody therapy aims at enhancing the subject immune response towards the cancer cells, notably by targeting cancer cells for destruction, by stimulating the activation of T cells present in the tumor or by preventing the inhibition of T cells present in the tumor, or at inhibiting the growth or spreading of cancer cells.

As used herein, “antibody therapy” may include the administration of monoclonal antibodies, polyclonal antibodies, multiple-chain antibodies, single-chain antibodies, single-domain antibodies, antibody fragments, antibody domains, antibody mimetics or multi-specific antibodies such as bispecific antibodies.

In one embodiment, the antibody is for or aims at targeting cancer cells or tumor cells for destruction.

Examples of antibodies, in particular monoclonal antibodies, targeting cancer cells or tumor cells for destruction include tumor-specific antibodies, in particular tumor-specific monoclonal antibodies. Examples of tumor-specific antibodies, include, without being limited to, antibodies targeting cell surface markers of cancer cells or tumor cells, antibodies targeting proteins involved in the growth or spreading of cancer cells or tumor cells.

In one embodiment, the antibody is for or aims at stimulating the activation of T cells present in the tumor.

Examples of antibodies, in particular monoclonal antibodies, stimulating the activation of T cells present in the tumor include, without being limited to, anti-CD137 antibodies and anti-OX40 antibodies as described hereinabove.

In one embodiment, the antibody is for or aims at preventing the inhibition of T cells present in the tumor.

Examples of antibodies, in particular monoclonal antibodies, preventing the inhibition of T cells present in the tumor include, without being limited to, anti-PD-1 antibodies (such as pembrolizumab, nivolumab, cemiplimab, tislelizumab, and spartalizumab), anti-PD-L1 antibodies (such as avelumab, atezolizumab, and durvalumab) and anti-CTLA-4 antibodies (such as ipilimumab and tremelimumab) as described hereinabove.

In one embodiment, the antibody is for or aims at inhibiting the growth or spreading of cancer cells.

Examples of antibodies inhibiting the growth or spreading of cancer cells include, without being limited to, anti-HER2 antibodies (such as trastuzumab).

As used herein, the term “vaccination” refers to the use of a preparation comprising a substance or a group of substances (i.e., a vaccine) meant to induce and/or enhance in a subject a targeted immune response towards cancer cells. Prophylactic vaccination is used to prevent a subject from ever having a particular disease or to only have a mild case of the disease. Therapeutic vaccination is intended to treat a particular disease in a subject, for example cancers. For example, therapeutic anti-cancer vaccines may comprise a tumor-associated antigen or tumor-associated antigens, aiming at inducing and/or enhancing a cell-mediated immune response, in particular a T cell immune response, directed towards the cancer cells expressing said tumor-associated antigen(s).

As used herein, a “therapeutic vaccine” is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; RNA-based vaccines, and dendritic cell vaccines.

In some embodiments, the primary or secondary treatment comprises the administration of an agent that increases the expression of alpha-2 adrenergic receptors, preferably at the RNA level. As used herein, the expression “increases the expression of alpha-2 adrenergic receptors” is intended to mean that the agent promotes the synthesis of alpha-2 adrenergic receptors, which results in an increased number of alpha-2 adrenergic receptors in the presence of said agent. Illustratively, the increase may represent at least about 10% increase of the number of alpha-2 adrenergic receptors.

Within the scope of the invention, the expression “about 10%” encompasses about 10%, 20%, 30%, 40%, 50%; 60%, 70%, 80%, 90%, 100%, 150%, 200%, 250%, 300%, 350%, 400%, 450%, 500%, 550%, 600%, 650%, 700%, 750%, 800%, 850%, 900%, 950%, about 1,000%, or more. In practice, the increase in the number of alpha-2 adrenergic receptors may be assessed by any suitable methods, or a method adapted therefrom, in particular immunostaining, western blotting, and the like.

In certain embodiments, the agent that increases the expression of alpha-2 adrenergic receptors may be an activating RNA.

In certain embodiments, said primary or secondary treatment is to be administered separately or concomitantly with said agonist.

Another aspect of the invention relates to a pharmaceutical composition comprising an alpha-2 adrenergic receptor agonist, as defined in the instant invention, and a pharmaceutically acceptable vehicle, for use in the prevention and/or the treatment of a spleen disorder.

Pharmaceutically acceptable excipients that may be used in the pharmaceutical composition of the invention include, without being not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene- polyoxypropylene- block polymers, polyethylene glycol, wool fat, and the like.

The instant invention further pertains to a combination kit comprising (i) an alpha-2 adrenergic receptor agonist or a pharmaceutical composition comprising an alpha-2 adrenergic receptor agonist and (ii) a therapeutic agent selected in the group consisting of an antibiotic, an antiparasitic, an antiviral, a chemotherapy agent, an immunotherapy agent, and the like, for use for the prevention and/or the treatment of a spleen disorder.

In some aspect, the invention also relates to the use of an alpha-2 adrenergic receptor agonist for the preparation and/or the manufacture of a medicament for the prevention and/or the treatment of a spleen disorder. The invention further relates to the use of an alpha-2 adrenergic receptor agonist for the preparation and/or the manufacture of a medicament for the treatment of a spleen disorder.

Another aspect of the invention relates to the use of an alpha-2 adrenergic receptor agonist for the prevention and/or the treatment of a spleen disorder. The invention also relates to the use of an alpha-2 adrenergic receptor agonist for the treatment of a spleen disorder.

In one aspect, the invention relates to a method for the prevention and/or the treatment of a spleen disorder in an individual in need thereof comprising the administration of a therapeutically efficient amount of an alpha-2 adrenergic receptor agonist. The invention further relates to a method for the treatment of a spleen disorder in an individual in need thereof comprising the administration of a therapeutically efficient amount of an alpha-2 adrenergic receptor agonist.

Another aspect of the invention relates to a method for reducing the volume and/or the weight of a spleen in an individual in need thereof comprising the administration of a therapeutically efficient amount of an alpha-2 adrenergic receptor agonist.

### BRIEF DESCRIPTION OF THE DRAWINGS

FIG. 1 is a histogram showing that alpha-2 adrenergic receptor agonist guanabenz can reverse the tumor-induced splenomegaly in TiRP tumor bearing mice. TiRP tumor-bearing mice (black bars and light grey bars) received daily injections of vehicle control (black bars) or guanabenz (5 mg/kg, i.p.; light grey bars) when the tumor size was around 500 mm³, until the day of sacrifice. As compared to non-tumor bearing mice (dark grey bars), mice bearing TiRP tumor developed splenomegaly which was reflected in a significant increased spleen weight (black bars). Following guanabenz administration (light grey bars), the weight of enlarged spleens was decreased. The weight of the spleen is expressed in grams (g).

FIG. 2 is a histogram showing that TiRP tumor-induced splenomegaly is inhibited by clonidine and romifidine, two alpha-2 adrenaline receptor agonists. TiRP tumor bearing mice received daily injections of clonidine (5 mg/kg, i.p.; Clonidine), romifidine (5 mg/kg, i.p.; Romifidine) or PBS (Con) when the tumor size was around 500 mm³. Mice were sacrificed when the tumor in control group reach 1,500 mm³. Spleens were isolated and weighted. Spleen from mice of same age without tumors were used as controls (non tumor Con). The weight of the spleen is expressed in grams (g). * P<0.05, ** P<0.01.

FIG. 3A-B are histograms showing that alpha-2 adrenergic receptor agonist guanabenz can reverse the tumor-induced splenomegaly in B16F1 tumor-bearing mice (A) and B16F10 tumor-bearing mice (B). Mice received daily injections of guanabenz (5 mg/kg, i.p.) or the vehicle control when the tumor size was around 100 mm³, until the day of sacrifice. As compared to non-tumor bearing mice, mice bearing B16F1 tumor developed splenomegaly which was reflected in a significant increased spleen weight. Following guanabenz administration, the weights of the enlarged spleens were decreased (A). Same observation was found for B16F10 tumor-bearing mice (B). The weight of the spleen is expressed in grams (g). * P<0.05.

FIG. 4 is a histogram showing that clonidine treatment reduced spleen size in the tumor-bearing mice, and this effect was attenuated when clonidine was combined with phentolamine, an alpha-2 adrenergic receptor antagonist, indicating that clonidine inhibits splenomegaly via its agonistic effect on the alpha-2 adrenergic receptor. TiRP-tumor bearing mice received adoptive cell transfer (ACT) of 10 million P1A-specific activated CD8+ T cells and daily injections of vehicle control, clonidine (5 mg/kg, i.p.), or clonidine (5 mg/kg, i.p.) plus phentolamine (5 mg/kg, i.p.), when the tumor size was around 500 mm³, until the day of sacrifice. The weight of the spleen is expressed in grams (g).

FIG. 5 is a histogram showing that clonidine treatment reduced the spleen size of the tumor-bearing mice, and this effect was attenuated when combined with phentolamine, an alpha-2 adrenergic receptor antagonist, indicating that clonidine inhibits splenomegaly via its agonistic effect on alpha-2 adrenergic receptor. In contrast, propranolol, a beta-adrenergic receptor antagonist, did not revert the effect of clonidine on splenomegaly. BALB/c mice bearing CT26 colon carcinomas received daily injections of vehicle control, clonidine (5 mg/kg, i.p.), clonidine (5 mg/kg, i.p.) plus phentolamine (5 mg/kg, i.p.), clonidine (5 mg/kg, i.p.) plus propranolol (5 mg/kg, i.p.), phentolamine (5 mg/kg, i.p.), or propranolol (5 mg/kg, i.p.), when the tumor size was around 50 mm³, until the day of sacrifice. The weight of the spleen is expressed in grams (g). * P<0.05, *** P<0.001.

FIG. 6 is a histogram showing that guanabenz treatment reduced the spleen size of the tumor-bearing mice, and this effect was attenuated when combined with phentolamine, an alpha-2 adrenergic receptor antagonist, indicating that guanabenz inhibits splenomegaly via its agonistic effect on alpha-2 adrenergic receptor. BALB/c mice bearing CT26 colon carcinomas received daily injections of vehicle control, phentolamine (5 mg/kg, i.p.), guanabenz (5 mg/kg, i.p.), or guanabenz (5 mg/kg, i.p.) plus phentolamine (5 mg/kg, i.p.), when the tumor size was around 50 mm³, until the day of sacrifice. The weight of the spleen is expressed in grams (g). ** P<0.01, *** P<0.001.

FIG. 7 is a histogram showing that guanabenz inhibits splenomegaly induced by a virus-mediated (Chadox_li_P1A (prime), MVA_P1A (boost)) immunization. The weight of the spleen is expressed in grams (g). ** P<0.01.

FIG. 8 is a histogram showing that guanabenz inhibits splenomegaly induced by protein-mediated (Ovalbumin) immunization. The weight of the spleen is expressed in grams (g). ** P<0.01.

FIG. 9 is a histogram showing that clonidine or guanabenz administration significantly prevents splenomegaly induced by myelofibrosis (induced by JAK2V617F mutated bone marrow transplantation), as measured by the spleen weight (g). One week after bone marrow transplantation, mice were treated with guanabenz or clonidine at a dose of 5 mg/kg daily for three weeks. After three weeks of treatment, mice were dissected and the spleen weight was measured. Wildtype mice that did not receive bone marrow transplantation or clonidine/guanabenz treatment were used as controls.

FIG. 10 is a histogram showing that clonidine or guanabenz administration significantly provides a treatment to splenomegaly induced by myelofibrosis (induced by JAK2V617F mutated bone marrow transplantation), as measured by the spleen weight (g). Three weeks after bone marrow transplantation, mice were treated with guanabenz or clonidine at a dose of 5 mg/kg daily for two weeks. After two weeks of treatment, mice were dissected and the spleen weight was measured. Wildtype mice that did not receive bone marrow transplantation or clonidine/guanabenz treatment were used as controls.

FIG. 11 is a histogram showing that clonidine treatment significantly reduces splenomegaly, as measured by the spleen weight (g), in romiplostim-mediated myelofibrosis. ** P<0.01; ns: not significant.

FIG. 12 is a histogram showing that clonidine or guanabenz inhibit tumor-induced splenomegaly, as measured by the spleen weight (g) in wild type mice (black bars). The inhibition of splenomegaly is abolished in Adra2a KO mice (grey bars). Asterisk indicates statistical relevance (P<0.05).

### EXAMPLES

The present invention and disclosure are further illustrated by the following examples.

### Example 1: Materials and Methods

1—Mice

TiRP mice have been created by crossing Ink4a/Arf^{flox/flox} mice with mice carrying a transgenic construct controlled by the tyrosinase promoter and driving the expression of H-Ras^{12V} and Trap1a which encodes a MAGE-type tumor antigen P1A; the promoter is separated from the coding region by a stop cassette made of a floxed self-deleting CreER (Huijbers et al., 2006, Cancer Res 66, 3278-3286). Those mice were backcrossed to a B10.D2 background and bred to homozygosity. TCRP1A mice heterozygous for the H-2Ld/P1A35-43-specific TCR transgene were kept on the B10.D2;Rag1^{-/-} background (Shanker et al., 2004, J Immunol 172, 5069-5077). DBA/2 mice and C57BL/6J mice were used in tumor transplantation and mice immunization experiments. All mice used in this study were produced under specific pathogen free (SPF) conditions at the animal facility of the de Duve Institute. All the rules concerning animal welfare have been respected according to the 2010/63/EU Directive. All procedures were performed with the approval of the local Animal Ethical Committee, with reference 2015/UCL/MD/15.

2—Tumor Induction with 40H-Tamoxifen

A fresh solution of 40H-Tamoxifen was prepared by dissolving 40H-Tamoxifen (Imaginechem®) in 100% ethanol and mineral oil (ratio 1:9) followed by 30 min sonication, and injected subcutaneously (2 mg/200 μL per mouse) in the neck area of gender-matched 7 weeks old TiRP mice. Tumor appearance was monitored daily and tumors were measured three times/week. Tumor volume (in mm³) was calculated by the following formula: Volume=width²×length/2.

3—Cell Cultures

B16F1, B16F10 melanoma cells, and CT26 colon carcinoma cells were cultured in IMDM medium supplemented with 10% Fetal bovine serum (FBS).

4—Adoptive Cell Transfer with TCRP1A CD8+ T Cells

For the adoptive cell transfer (ACT), P1A-specific (TCRP1A) CD8+ T cells were isolated from spleens and lymph nodes of TCRP1A mice as described hereinabove, and stimulated in vitro by co-culture with irradiated (10,000 rads) L1210.P1A.B7-1 cells (Gajewski et al., 1995, J Immunol 154, 5637-5648) at 1:2 ratio (0.5×10⁵ CD8+ T cells and 10⁵ L1210.P1A.B7-1 cells per well in 48-well plates) in IMDM (GIBCO®) containing 10% fetal bovine serum supplemented with L-arginine (0.55 mM, Merck®), L-asparagine (0.24 mM, Merck®), glutamine (1.5 mM, Merck®), beta-mercaptoethanol (50 μM, Sigma Aldrich®), 50 UmL⁻¹ penicillin and 50 mg mL⁻¹ streptomycin (Life Technologies®). Four days later, TCRP1A CD8⁺ T cells were purified on a Lymphoprep gradient (StemCell®) and 10⁷ living cells were injected intravenously in 200 μL PBS in TiRP-tumor bearing mice on the day of randomization.

5—Evaluation of Effect of Alpha-2 Adrenaline Receptor Agonists on Tumor Induced Splenomegaly

C57BL/6 mice were injected subcutaneously with 1 million B16F1 or B16F10 melanoma cells at an age between 6 to 8 weeks. The mice received daily injections of guanabenz (5 mg/kg, i.p.) or PBS when the tumor size was around 100 mm³. Mice were sacrificed when the tumor in control group reached 1,000 mm³. Spleen were isolated and weighted. Spleen from mice of same age that did not receive tumor implantation were used as controls. TiRP tumor bearing mice received daily injections of clonidine (5 mg/kg, i.p.), romifidine (5 mg/kg, i.p.) or PBS when the tumor size was around 500 mm³. Mice were sacrificed when the tumor in control group reached 1,500 mm³. Spleen were isolated and spleen weight was measured with an analytical balance. Spleen from mice of same age that did not have tumors but received ACT were used as controls.

6—Evaluation of Alpha-2 Adrenaline Receptor Agonists/Antagonists in Tumor-Bearing Mice
a) TiRP Tumor Model

TiRP-tumor bearing mice received adoptive cell transfer (ACT) of 10 million P1A-specific activated CD8+ T cells and daily injections of vehicle control, clonidine (5 mg/kg, i.p.), or clonidine (5 mg/kg, i.p.) combined with phentolamine (5 mg/kg, i.p.), when the tumor size was around 500 mm³, until the day of sacrifice. Tumor size was monitored every 2 days. Mice were sacrificed when the tumor in control group reached around 2,000 mm³. Spleen were isolated and spleen weight was measured with an analytical balance. Spleen from mice of same age without tumors but received ACT were used as controls.
b) CT26 Tumor Model

C57BL/6 mice were injected subcutaneously with 2 million CT26 colon tumor cells at an age of 6 to 8 weeks. For evaluation of phentolamine on the effect of clonidine and guanabenz, the mice were randomized into different groups when tumors size arrived around 50 mm³ and received daily injections of guanabenz (5 mg/kg, i.p.), phentolamine (5 mg/kg, i.p.), propranolol (5 mg/kg, i.p.), a mix of guanabenz (5 mg/kg, i.p.) and phentolamin (5 mg/kg, i.p.), clonidine (5 mg/kg, i.p.), a mix of clonidine (5 mg/kg, i.p.) and phentolamin (5 mg/kg, i.p.), a mix of clonidine (5 mg/kg, i.p.) and propranolol (5 mg/kg, i.p.) or vehicle control. Tumor size was measured every 2 days and mice were sacrificed when the tumor in control group reached 1,000 mm³. Spleen were isolated and spleen weight was measured with an analytical balance. Spleen from mice of same age without tumors were used as controls.

7—Evaluation of Effect of Alpha-2 Adrenaline Receptor Agonists on Immunization Induced Splenomegaly
a) Immunization with Ovalbumin

C57BL/6J mice were immunized once by intraperitoneal (i.p.) injection of 200 μg of OVA protein adsorbed onto Alhydrogel adjuvant 2% (Sigma Aldrich®). These mice were administered 100 μg (5 mg/kg) of guanabenz 2 hours before the immunization and daily after the immunization. Mice that did not receive immunization were included as negative control. One week after the immunization, mice were sacrificed, the spleen of each mouse was collected, and spleen weight was measured with an analytical balance.

b) Immunization with Viral Vector

DBA/2 mice were immunized by intramuscular (i.m) injection of Chadox_li_P1A (a chimpanzee adenoviral vector recombinant for tumor antigen P1A; 10⁷ IU, prime) followed by second injection (i.m) of MVA_P1A (a modified vaccinia Ankara viral vector recombinant for tumor antigen P1A; 10⁶ IU, boost) one week after. These mice were administered 100 μg (5 mg/kg) of guanabenz 2 hours before the immunization and daily after the immunization. Mice that did not receive immunization were included as negative control. Two weeks after the immunization, mice were sacrificed, the spleen of each mouse was collected, and spleen weight was measured with an analytical balance.

### Example 2: Effect of Guanabenz, Clonidine or Romifidine Treatment on the Weight of the Spleen of TiRP Tumor-Bearing Mice

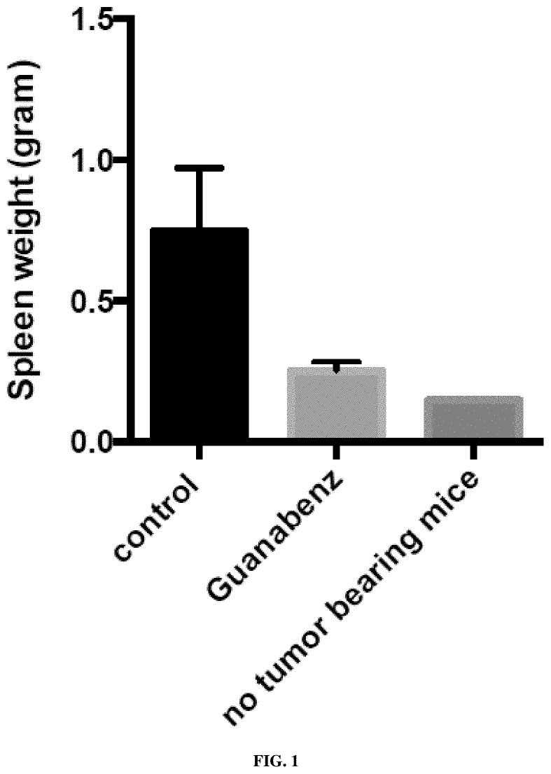

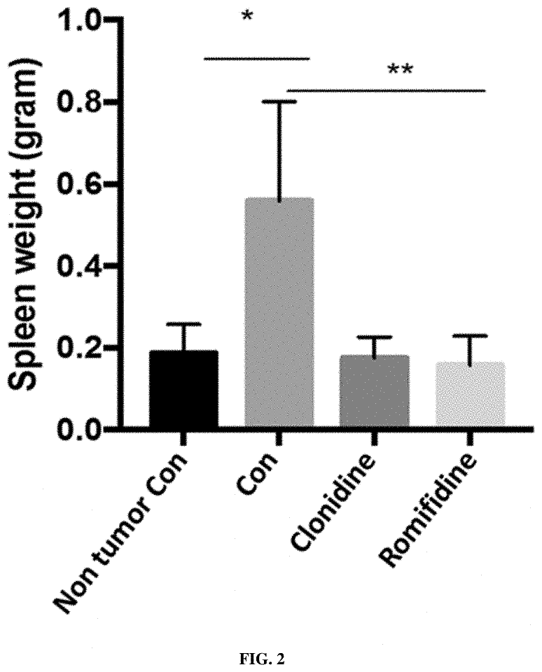

FIG. 1 shows that the alpha-2 adrenergic receptor agonist guanabenz can reverse the tumor-induced splenomegaly in TiRP tumor bearing mice. The same observations were made for clonidine and romifidine (FIG. 2).

### Example 3: Effect of Guanabenz Treatment on the Weight of the Spleen of B16F1 or B16F10 Tumor-Bearing Mice

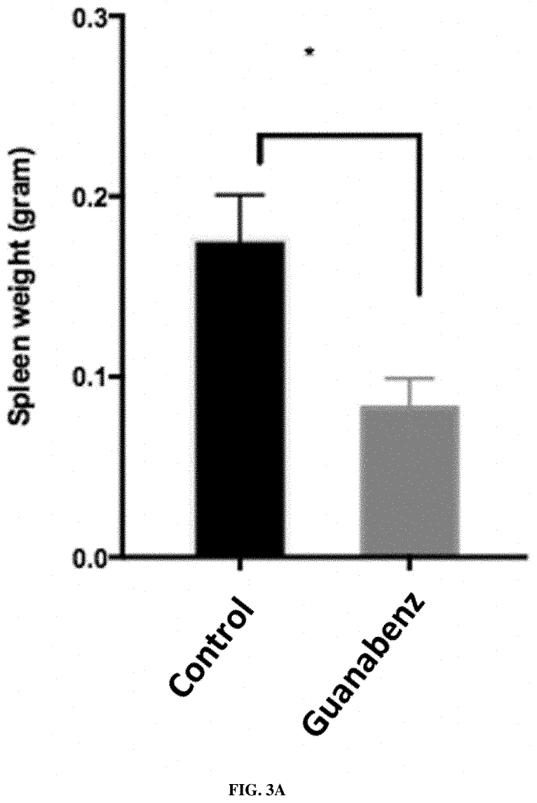

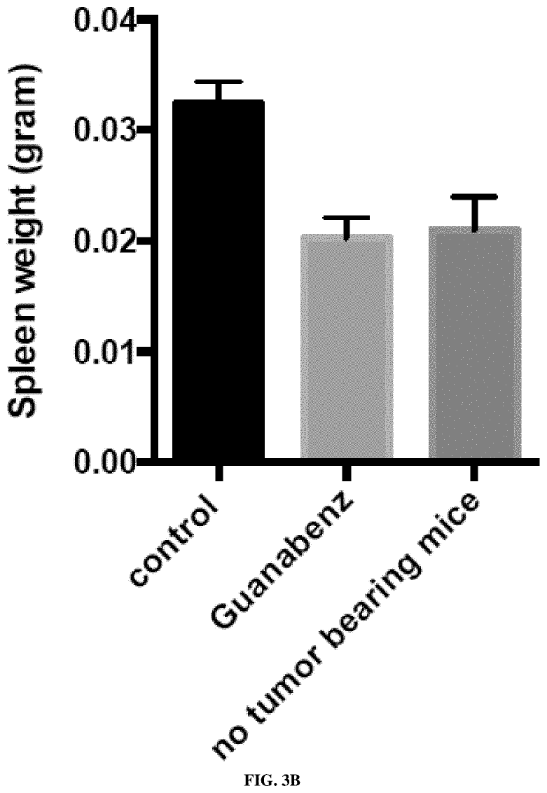

FIG. 3A-B shows that alpha-2 adrenergic receptor agonist guanabenz can reverse the tumor-induced splenomegaly in B16F1 tumor-bearing mice (panel A) and B16F10 tumor-bearing mice (panel B).

### Example 4: Effect of the Combined Treatment of Adoptive Cell Transfer (ACT) and Clonidine on the Weight of the Spleen of TiRP Tumor-Bearing Mice

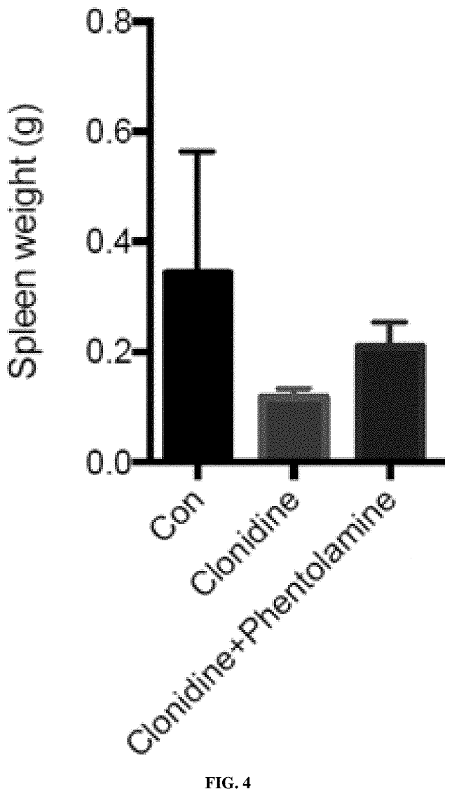

FIG. 4 shows that clonidine treatment reduced spleen size in the tumor-bearing mice, and this effect was attenuated when clonidine was combined with phentolamine, an alpha-2 adrenergic receptor antagonist, indicating that clonidine inhibits splenomegaly via its agonistic effect on the alpha-2 adrenergic receptor.

### Example 5: Effect of Clonidine or Guanabenz Treatment on the Weight of the Spleen of BALB/c Mice Bearing CT26 Colon Carcinomas

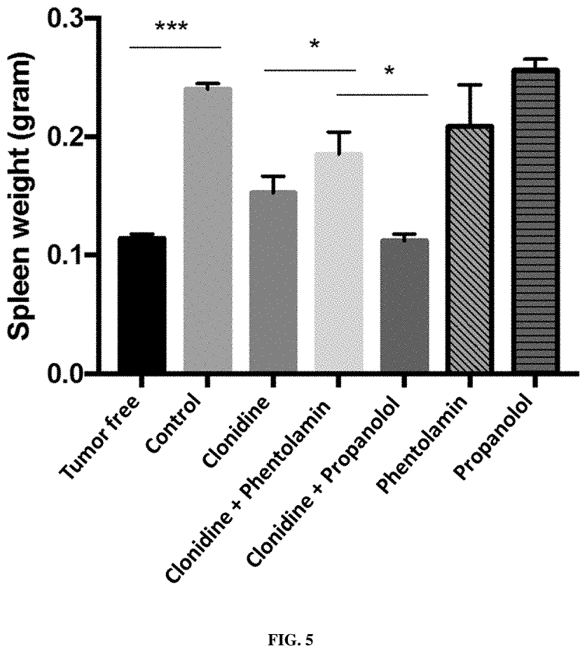

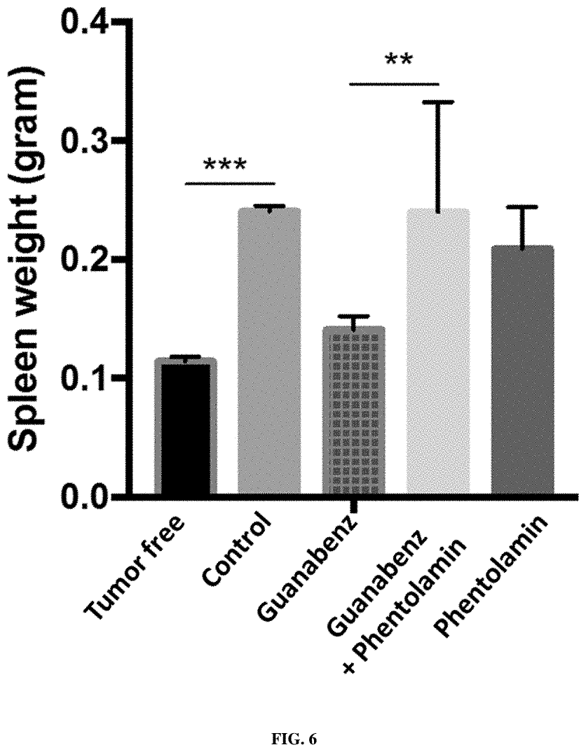

Clonidine treatment (FIG. 5) and guanabenz treatment (FIG. 6) are capable of reducing the spleen size of the tumor-bearing mice. This effect was attenuated when combined with phentolamine, an alpha-2 adrenergic receptor antagonist, indicating that clonidine and guanabenz inhibit splenomegaly via their agonistic effect on alpha-2 adrenergic receptor.

### Example 6: Clonidine Inhibits Splenomegaly Induced by Virus-Mediated or Protein-Mediated Immunization

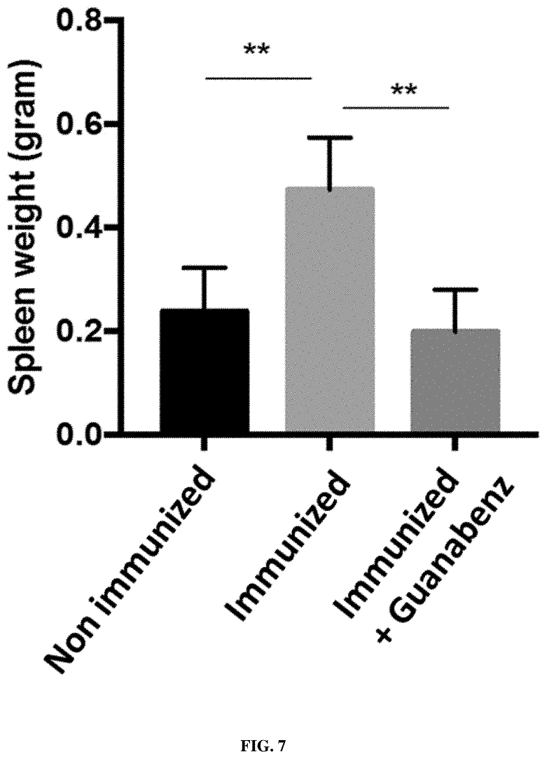

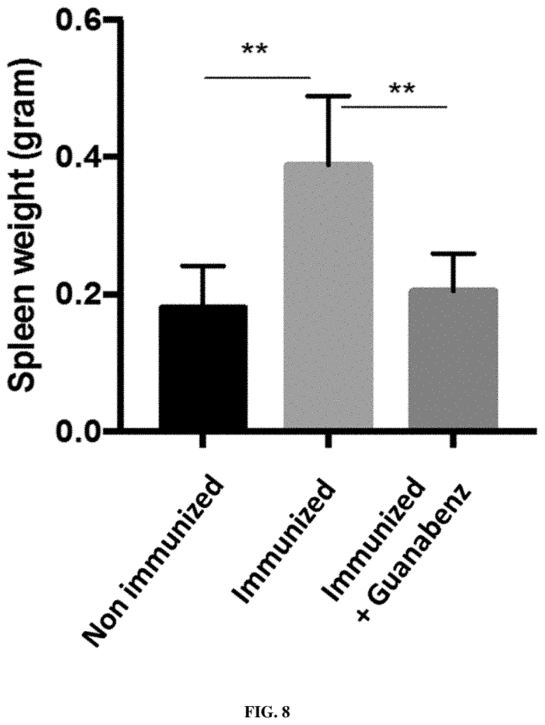

Guanabenz treatment is efficiently capable of reducing the spleen size of mice which have splenomegaly resulting from virus-mediated (FIG. 7) or protein-mediated (FIG. 8) immunization.

### Example 7: Clonidine or Guanabenz Inhibits Splenomegaly Induced by Myelofibrosis (1^{st} Model)

Myelofibrosis (MF) is a clonal malignant disease resulting from acquisition of JAK-STAT activating driver mutations, of which JAK2V617F is the most prevalent. Splenomegaly is one of the major clinical manifestations of MF and is directly linked to splenic extramedullary hematopoiesis.

Murine femurs and tibias from JAK2V617F mutant mice were first harvested and cleaned thoroughly. Marrow cells were flushed into PBS with 2% fetal bovine serum using a 25G needle and syringe. Resulting cell suspensions were then filtered through a 40 uM cell strainer. Recipient mice were irradiated with two doses of 500 rad 4 h apart. 1 million of donor cells were injected into wild-type recipients by standard intravenous injection using a 27 G insulin syringe.

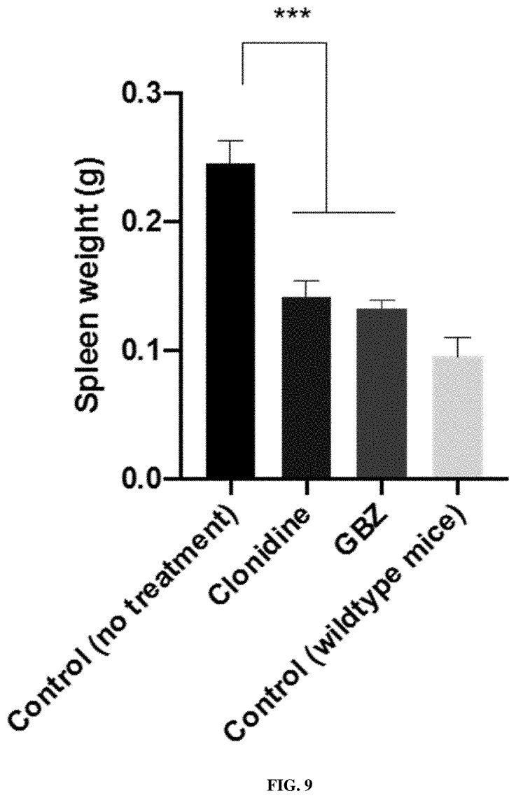

In a first alternative, one week after bone marrow transplantation, mice were treated with guanabenz or clonidine at a dose of 5 mg/kg daily for three weeks. After three weeks of treatment, mice were dissected and the spleens weight were measure. Wildtype mice that did not receive bone marrow transplantation or clonidine/guanabenz treatment were used as controls. As shown in FIG. 9, both clonidine and guanabenz treatments significantly reduced the splenomegaly induced by JAK2V617F mutated bone marrow.

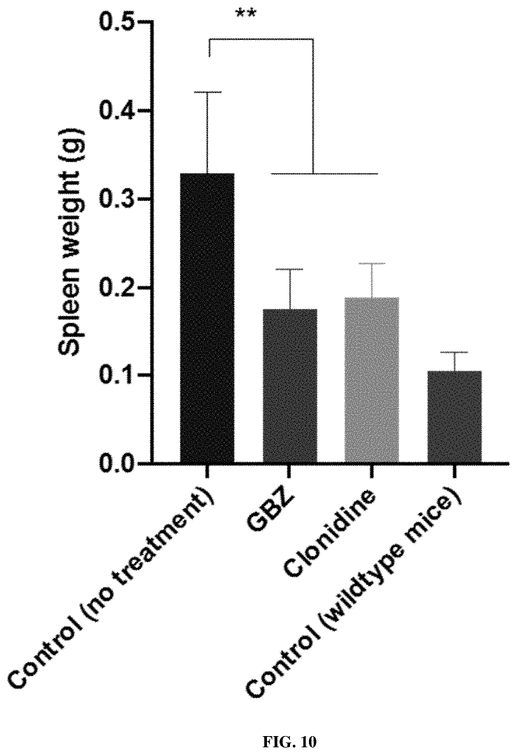

In a second alternative, three weeks after bone marrow transplantation, mice were treated with guanabenz or clonidine at a dose of 5 mg/kg daily for two weeks. After two weeks of treatment, mice were dissected and the spleens weight were measure. Wildtype mice that did not receive bone marrow transplantation or clonidine/guanabenz treatment were used as controls. In other words, the drug was given 3 weeks after bone marrow transplantation when the splenomegaly already formed. As shown in FIG. 10, both clonidine and guanabenz treatments significantly reduced the splenomegaly induced by JAK2V617F mutated bone marrow. This is indicating that the drug can be used not only for prevention but also for therapeutic purpose.

### Example 8: Clonidine or Guanabenz Inhibits Splenomegaly Induced by Myelofibrosis (2^{nd} Model)

Romiplostim is a ligand binding to the Thrombopoietin Receptor (TPO) Ligand. Recent studies have revealed that thrombopoietin (TPO)/myeloproliferative leukemia protein (MPL; TPO receptor) signaling pathway play a certain role in the development of Myelofibrosis (MF). Myeloproliferative leukemia protein activation directly induces fibrocyte differentiation to cause myelofibrosis. Administration of TPO ligand induced MF and splenomegaly.

8-week female C57BL/6 J wild-type and Adra2a KO mice were administered saline or 1 mg/kg of romiplostim by a subcutaneous injection into the neck skin on days 1, 8 and 15. Clonidine or guanabenz were administrated i.p. at a dose of 5 mg/kg daily for two weeks starting from day 8. Mice were dissected on day 22 and spleen weight were measured. After killing, peripheral blood was drawn by cardiac puncture, the platelet concentration was measured by Cell Counter Analyzer MS9-5V.

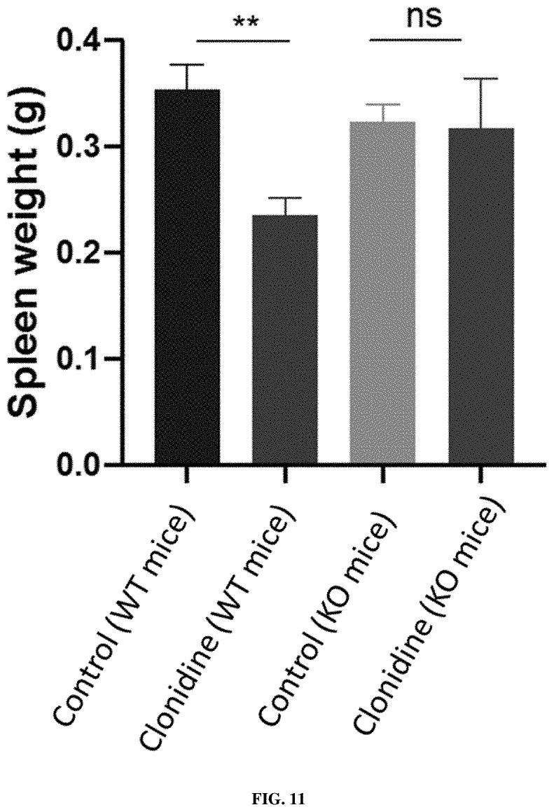

As shown in FIG. 11, clonidine treatment significantly reduced the splenomegaly induced by TPO receptor activation. This happens only in the wild-type mice but not in Adra2a KO mice, indicating that the inhibition of splenomegaly is via adrenergic receptor alpha-2.

### Example 9: Clonidine and Guanabenz Inhibit Tumor-Induced Splenomegaly

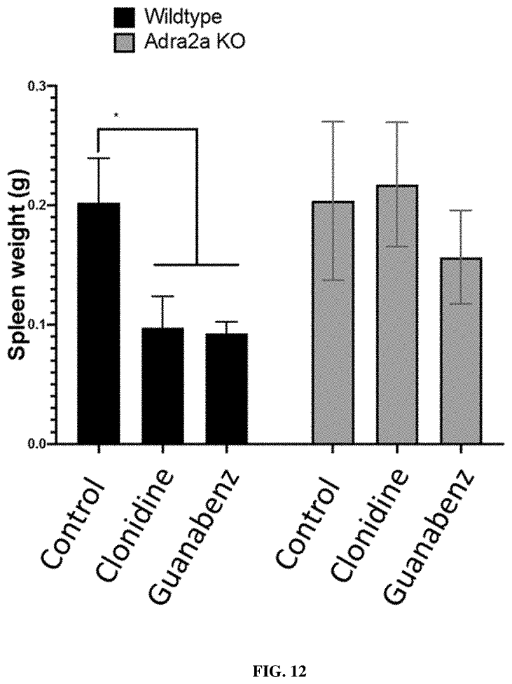

C56BL/6 Wildtype or C56BL/6 Adra2a KO mice were implanted with MC38 colon carcinomas and received daily injections of vehicle control, guanabenz or clonidine (5 mg/kg, i.p.) when the tumor size was around 50 mm³, until the day of sacrifice. Spleen weight were taken at the time of mice dissection. As shown in FIG. 12, clonidine and guanabenz treatments inhibit tumor-induced splenomegaly. This is via adrenergic receptor alpha-2 since no inhibition is observed in the Adra2a KO mice.

1-17. (canceled)

18. A method for the prevention and/or the treatment of a spleen disorder of the spleen in an individual in need thereof comprising the administration of a therapeutically efficient amount of an alpha-2 adrenergic receptor agonist.

19. The method according to claim 18, wherein said spleen disorder is splenomegaly.

20. The method to claim 18, wherein said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

21. The method according to claim 18, wherein said agonist is selected in the group consisting of brimonidine, clonidine, dexmedetomidine, guanabenz, guanfacine, lofexidine, methyldopa, romifidine, tizanidine, xylazine, and a functional derivative thereof, in particular clonidine, guanabenz, romifidine, or a functional derivative thereof.

22. The method according to claim 18, wherein said agonist is selected in the group consisting of an antibody, an antibody fragment, an afucosylated antibody, a diabody, a triabody, a tetrabody, a nanobody, and an analog thereof.

23. The method according to claim 18, wherein said agonist does not cross the blood/brain barrier.

24. The method according to claim 18, wherein said agonist is administered at a dose ranging from about 0.0001 mg/kg body weight to about 100 mg/kg body weight.

25. The method according to claim 18, wherein said spleen disorder is associated with a disorder selected in the group consisting of a hypersplenism, cancer, liver disorder, cystic fibrosis, an inflammatory disease, a microbial infection, a hemolytic anemia, and the like.

26. The method according to claim 18, wherein said spleen disorder is associated with blood cancer or solid cancer.

27. The method according to claim 18, wherein said agonist is administered with a primary or a secondary treatment selected in the group consisting of antimicrobial agents, anti-inflammatory agents, chemotherapy, immunotherapy, radiation, the like, and a combination thereof.

28. The method according to claim 27, wherein said immunotherapy comprises adoptive transfer of immune cells, a checkpoint inhibitor, vaccination, the like, and a combination thereof.

29. The method according to claim 27, wherein said immunotherapy comprises adoptive transfer of immune cells selected in the group comprising T cells, in particular CD8+ T cells and CAR T cells; natural killer (NK) cells, in particular CAR NK cells; the like; and a combination thereof.

30. The method according to claim 27, wherein said primary or secondary treatment is administered separately or concomitantly with said agonist.

31. A combination kit comprising (i) an alpha-2 adrenergic receptor agonist or a pharmaceutical composition comprising an alpha-2 adrenergic receptor agonist and (ii) a therapeutic agent selected in the group consisting of an antibiotic, an antiparasitic, an antiviral, a chemotherapy agent, an immunotherapy agent, and the like, for the prevention and/or the treatment of a spleen disorder.

32. A method for reducing the volume and/or the weight of a spleen in an individual in need thereof comprising the administration of a therapeutically efficient amount of an alpha-2 adrenergic receptor agonist.

33. The method to claim 32, wherein said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

34. The method according to claim 32, wherein said agonist is selected in the group consisting of brimonidine, clonidine, dexmedetomidine, guanabenz, guanfacine, lofexidine, methyldopa, romifidine, tizanidine, xylazine, and a functional derivative thereof, in particular clonidine, guanabenz, romifidine, or a functional derivative thereof.

35. The method according to claim 32, wherein said agonist is selected in the group consisting of an antibody, an antibody fragment, an afucosylated antibody, a diabody, a triabody, a tetrabody, a nanobody, and an analog thereof.

36. The method according to claim 32, wherein said agonist does not cross the blood/brain barrier.
